(19) United States
(12) Patent Application Publication
(10) Pub. No.: US 2020/0315523 A1
(43) Pub. Date: Oct. 8, 2020

(54) SYSTEM AND METHOD FOR A WEARABLE KNEE INJURY PREVENTION

(71) Applicant: Andrew Tesnow, Springfield, VA (US)

(72) Inventor: Andrew Tesnow, Springfield, VA (US)

(21) Appl. No.: 16/839,314

(22) Filed: Apr. 3, 2020

Related U.S. Application Data
(60) Provisional application No. 62/829,473, filed on Apr. 4, 2019.

(51) Int. Cl. A61B 5/00; A61B 5/15

(52) U.S. Cl. CPC A61B 5/4585; A61B 5/4533; A61B 5/6828; A61B 5/0022; A61B 5/150809; A61B 5/150816; A61B 5/150824; A61B 5/746

(57) ABSTRACT

A method and system for actively monitoring anterior cruciate ligament (ACL) strain experienced at the knee joint during athletic activity or dynamic movement. Sensors are used in proximity of the knee joint to actively record parameters such as flexion angle and ground impact force at the knee joint. Sensor measurements are then inputted into a processing unit that will quantify a tibial shear force (TSF) value based on the sensor outputs and dynamically generate user feedback and/or warning signals when unsafe levels of TSF conducive to ACL injury are detected.

This application claims priority to U.S. Provisional Patent Application No. 62/829,473, entitled “SYSTEM AND METHOD FOR A WEARABLE KNEE INJURY PREVENTION” filed Apr. 4, 2019. The entire contents and disclosures of these patent applications are incorporated herein by reference in their entirety.

### CROSS-REFERENCE TO RELATED APPLICATIONS

This application makes reference to U.S. Provisional Patent Application No. 62/367,519, entitled, “WEARABLE DEVICE THAT MITIGATES THE PROBABILITY OF ACL TEAR BY WARNING AGAINST BAD FORM,” filed Jul. 27, 2016. The entire content and disclosure of this patent application is incorporated herein by reference in its entirety.

### BACKGROUND

### Field of the Invention

The present invention relates to assessment systems for preventing joint injury. More specifically, the present invention is directed to a system and method for preventing knee joint injuries during activity.

### Related Art

Many activities employ dynamic movements that may cause strain on the body. Excessive strain, for example, that applied to a disclosed bodily area may lead to negative impacts such as to the knee. Knee injuries, specifically anterior cruciate ligament (ACL) injuries, are fairly common and can have negative results that may last a long time.

ACL injuries can occur through both contact and non-contact means. Non-contact injuries may frequently occur. The main type of injuries is caused by a lack of flexion in an anatomical joint, such as the knee joint, or an increase in joint abduction. When knee flexion is low, the body actually works against itself by pulling on both ends of the ACL. Eventually, this causes the ACL to tear.

The anterior cruciate ligament (ACL) is a main stabilizer between the tibia and femur. Tearing it causes loss of mobility and may require surgery. There is a 13% chance for a National Collegiate Athletic Association athlete to tear their ACL in a given year. There are 300,000 ACL injuries a year within the United States alone. There is a large financial burden associated with an ACL tear. Each tear costs the medical health care system around $60 K per ACL and $2 k per patient. This cost includes, reconstruction, surgeries, anesthetics, equipment and reconstructive consumables (such as the screws that go into the leg). Moreover, there may be a rehabilitation cost to cover physical therapy services and equipment. In addition to the monetary burden, an ACL injury involves a substantial loss of time and quality of life for an individual. ACL tears may involve an average of 6 to 9 months of post-operation recovery period. However, some recoveries may take upwards of 12 months depending on the type of ACL graft. As for quality of life, approximately only 44% of athletes return to their pre-surgery level of athletic performance. This means that around 56% of athletes either do not return to play sports, or if they do, it is at a much lower level. Despite the associated cost in terms of money, time and quality of life, the identification and mitigation of the likelihood of these types of injuries remains faulty at best.

### SUMMARY

The forgoing needs are met, to a great extent, by the present invention wherein, according to a first broad aspect, the present invention provides a system comprising a first sensing unit for reading a measurement of impact force transmitted to an anatomical joint, into a processing unit. The system further comprises a second sensing unit for reading measurements of the anatomical joint flexion angle into the processing unit. Moreover, the system includes a warning system comprising of one or more user alert units coupled to the processing units. The processing unit computes one or more strain values experienced by the anatomical joint and activates the one or more user alert units when a detrimental strain threshold level is reached.

According to a second broad aspect, the present invention provides a method comprising measuring an instantaneous momentum of an anatomical limb at an instance of ground impact. The method further comprising a step of measuring a flexion angle of an anatomical joint coupled to the anatomical limb at the instance of ground impact and calculating one or more strain values experienced by the anatomical joint using as parameters the instantaneous momentum of the anatomical limb and the flexion angle of the anatomical joint at the instance of ground impact.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a structural illustration of a knee joint system, according to one embodiment of the present invention.

FIG. 2 is a stick diagram illustration of relevant angles and reference frames for calculating flexion-related strains at the knee joint, according to one embodiment of the present invention.

FIG. 3 is a graph illustrating muscle force contribution to Tibial Shear Force (TSF) as a function of the knee joint flexion angle, according to one embodiment of the present invention.

FIG. 4 is a graph illustrating vertical and horizontal ground reaction force contribution to TSF as a function of body weight, according to one embodiment of the present invention.

FIG. 5 is a schematic illustration of an identification and warning system for an ACL injury prevention system based on the TSF model, according to one embodiment of the present invention.

FIG. 6 is a schematic illustration of an ACL injury prevention system based on sensitivity analysis of TSF model, according to one embodiment of the present invention.

FIG. 7 is a block diagram representation of an ACL injury prevention system utilizing an accelerometer and a flexion angle sensor, according to one embodiment of the present invention.

FIG. 8 is a block diagram representation of an ACL injury prevention system utilizing a variable resistor sensor for measuring a flexion angle of the knee, according to one embodiment of the present invention.

FIG. 9 is an illustration of a knee brace with ACL injury prevention functionality, according to one embodiment of the present invention.

FIG. 10 is a block diagram representation of an anatomical joint injury prevention system utilizing a Bluetooth interface between sensor outputs and a specialized software application for processing the sensor output, in accordance to an embodiment of the invention.

FIG. 11 is a block diagram illustrating a method of determining and monitoring lower injury risk in humans using algorithms and a sensor configuration, in accordance to an embodiment of the invention.

FIG. 12 is a block diagram illustrating a system for determining and monitoring lower injury risks due to movement mechanics, in accordance to an embodiment of the invention.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present invention, the term “comprising”, the term “having”, the term “including,” and variations of these words are intended to be open-ended and mean that there may be additional elements other than the listed elements.

For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For the purposes of the present invention, the term “ACL” refers the anterior cruciate ligament (ACL) and may be regarded as one of the key ligaments that help stabilize the knee joint. The ACL connects the thighbone (femur) to the shinbone (tibia). It's most commonly torn during sports that involve sudden stops and changes in direction including, but not limited to, basketball, soccer, tennis and volleyball.

For the purposes of the present invention, the term “anatomical” refers the structure of an organism or any of its parts. It may also refer to the scientific study of the shape and structure of organisms and their parts, relating to the scientific study and representation of the physical body or relating to the structure of the body.

For the purposes of the present invention, the term “anatomical joint” refers to any joint connecting skeletal segments within human anatomy. Examples of anatomical joint are knee joint, elbow joint, hip joint, neck joint, etc.

For the purposes of the present invention, the term “Bluetooth®” refers to a wireless technology standard for exchanging data over short distances (using short-wavelength radio transmissions in the ISM band from 2400-2480 MHz) from fixed and mobile devices, creating personal area networks (PANs) with high levels of security. Created by telecom vendor Ericsson in 1994, it was originally conceived as a wireless alternative to RS-232 data cables. It can connect several devices, overcoming problems of synchronization. Bluetooth® is managed by the Bluetooth® Special Interest Group, which has more than 18,000 member companies in the areas of telecommunication, computing, networking, and consumer electronics. Bluetooth® was standardized as IEEE 802.15.1, but the standard is no longer maintained. The SIG oversees the development of the specification, manages the qualification program, and protects the trademarks. To be marketed as a Bluetooth® device, it must be qualified to standards defined by the SIG. A network of patents is required to implement the technology and are licensed only for those qualifying devices.

For the purposes of the present invention, the term “flexion angle” refers to the relative angle between two adjacent segment connected via a joint. For example a knee joint flexion angle refers to the relative angle between the femur and the shank.

For the purposes of the present invention, the term “limb” refers to an extremity as a jointed, or prehensile (as octopus arms or new world monkey tails), appendage of the human or other animal body. In the human body, the upper and lower limbs are commonly called the arms and the legs, respectively. It may also refer to a part or member of an animal or human body distinct from the head and trunk, as a leg, arm, or wing.

For the purposes of the present invention, the term “muscle memory” refers motor learning, which is a form of procedural memory that involves consolidating a specific motor task into memory through repetition.

For the purposes of the present invention, the term “NeuroTraining” refers to training using neuroplasticity, memory, and repetition of motion. NeuroTraining is the science of training muscle memory.

### DESCRIPTION

The disclosed embodiment facilitates addressing body injuries performing an action to accelerate itself against gravity. By observing such injuries, the disclosed invention is able to conduct a gap analysis between the current world and the perfect world. Disclosed embodiments close that gap. Current injury prevention methods in sports are constraint to the type of injury. For example, a knee injury from the knee abducting, or in other words, bending sideways, is best prevented by adding supporting structure to the knee in the form of a structured knee brace. On the other hand, a knee injury resulting from the leg's own force as it tries to stop its body's movement cannot be supported by a structure. The knee can only be trained not to hurt itself.

There are several injuries that result from this manner of miss-using the body's mechanism. The probability of getting injured by reacting against gravity and the body's mass can be lessened by training. If an athlete performs movements that are deemed safe repeatedly, then the athlete learns how to move safety. This is how neuroplasticity (learning) works with neuromuscular (cached movement memories or muscle memory). By learning how to move safety and updating the minds neuromuscular memory cache, the athlete will then use those safe movements with they are focused on something else like reacting to an opponent's advances, throwing a curveball or picking up a box. By performing safe movements within competition or a work environment, an athlete or worker will lower their probability of injury. Safe form is a part of good form. Good form is also form that is efficient and therefore enables a worker or athlete to perform their activity with less energy and therefore with better overall performance.

Muscle Imbalances are when the body's equilibrium in action is thrown off, because one muscle group is stronger than another and therefore the body's action is constraint by that imbalance. Injuries can occur by these imbalances, because that action that may or may not be safe is constraint to the muscles that enable it. If the muscles that enable the action cannot perform the action because it is too weak or another muscle system dominates it, then the action may be thrown off into bad form which can also be unsafe form that leads to lowered performance and even injury. Therefore, good form should be form that uses and trains the body's muscles to work in harmony as well as avoid actions that could lead to injury.

Another component of good form is efficient form. Efficient form is an action that the body takes to achieve a goal, like kicking a goal or swinging a golf club, by using the least amount of muscles required. By performing these movements with efficient form, an athlete or worker can then perform more repetitions of that action in good form which will allow them to be safer and longer. Good form is, therefore, safe, balanced, and efficient. By performing good form repeatedly, the body learns to be safer and more efficient which leads to better performance and the body adapts to imbalances by strengthening the weaker muscles which will balance the body's reactionary resources (muscles) and further increase safety and performance.

Currently, there are a few different ways to train good form. Coach training occurs when an athlete or worker in a group training environment is shown good form through examples and then is instructed to perform good form with observation from the coach or instructor. This can be effective, but there are certain draw backs. Group training greatly decreases the efficiency of correcting bad form, because there is one set of eyes on many people practicing the form. This can lead to athletes and workers performing bad form repeatedly without being corrected which is thereby learning bad form. Even the best coaches in the world cannot admit that they can correct all bad form occurrences of the whole group all the time. And the skill levels of form correcting by coaches varies greatly.

Personal training is when an athlete of worker has one on one personal time with a trainer or coach. This is more effective than coaches training, because a coach can watch exercises and correct movements whenever they see bad for occur. The draw back to this approach is that for learning and muscle adaption to occur, the athlete needs to perform certain exercises or movements many times to effectively learn. To achieve this will take lots of time from a personal trainer and that time is not always available. If it is available, then it will be expensive and may be cost prohibitive. A team environment is lacking in personal training and, therefore, may lose the environment of the activity which may have an effect on the results of the training.

Self-training is the last method and results from an athlete or worker practicing movements by themselves. A lone athlete can understand good form by remembering a class or period of instruction, or they can access videos and various types of media, or they can read and view diagrams. This results in a lone person trying to learn and understand a movement mentally while also trying to perform that move in free space. This is doubly taxing for the brain and this leads to errors in either understanding the form or paying attention to good form. Even if an athlete or worker has sufficiently learned good form, if they are performing high intensity workouts that involve cardio or muscle fatigue, their form can degrade into bad form and either lead to injury or enable the brain to learn and adapt to the bad fatigued form which could lead to injury at a later time.

Self-training however does have the advantage of being cheap and available to whenever the worker or athlete wishes to perform them. Many self-scheduling training regimens and guides exist but the effectiveness of these things is constraint on a user's input of repetitions and not analysis of good form. This does not ensure that good form is being practiced and therefore is not realizing the full potential of training regimens.

Disclosed embodiments of the present invention achieve good form by learning with all the effectiveness of personal training, the team environment of coach training and the personalized scheduling of self-training while maximizing the accuracy of form correction and minimizing cost. The disclosed embodiments may be implemented as part of a NeuroTraining system that steps up to the plate to fill this gap by providing constant form correction supervision to important athletic training movements at any time and at affordable cost. The NeuroTraining system also slows adaptive training schedules and routines based on a worker or athletes individual needs and goals. The NeuroTraining system is cost effective because it lowers the probability of debilitating costly injuries without the need for an expensive personal trainer and utilizes the full extent of a user's training effort in achieving the desired goal which is learning good form and training the muscles to be balanced and strong.

The NeuroTraining system has different components or modules that may be used separately or in a unified method to fully instruct a user to be a safer more efficient athlete or worker. In disclosed embodiments, separate or collective components of the NeuroTraineing System may connect to Smart Aps via Bluetooth to utilize computational power and memory to achieve accurate and persistent form analysis and then tailor a user's workout regimen and track their progress.

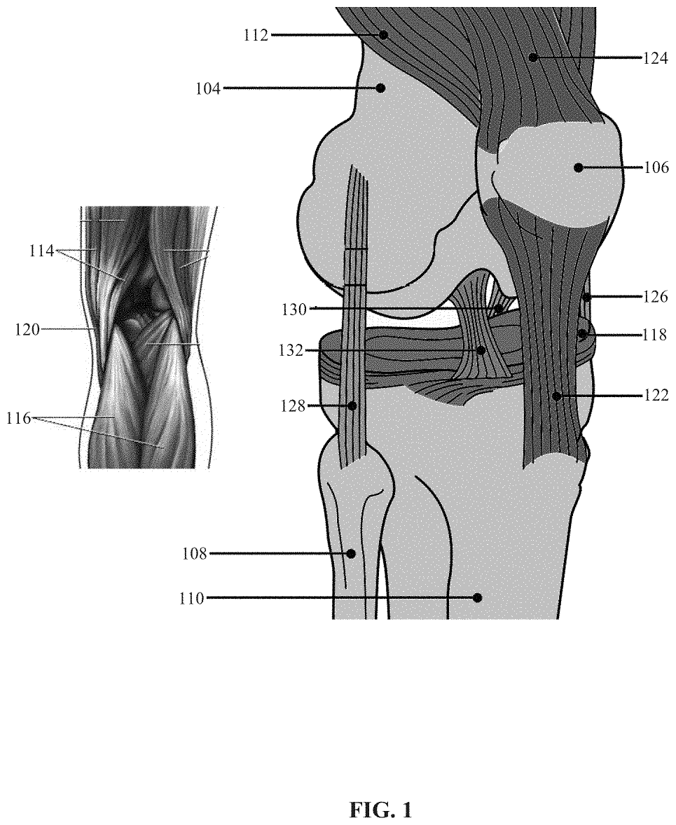

One component of the aforementioned NeuroTraining system may address possible knee injuries. The knee contains five major sets of components including bones, muscles, cartilages, tendons, and ligaments. As illustrated in FIG. 1, knee bones include the femur (104), patella (106), tibia (108), and fibia (110). The femur is the thigh bone. The fibia and tibia make up the shank, (the lower part of the leg) and the patella is also known as the knee cap. Muscles associated with the knee joint include the quadriceps (112), hamstrings (114), and gastrocnemius or calf muscles (116). The knee joint also includes a meniscus (118) which acts as a cushion to absorb shock during dynamic movements. The tendons in the knee joint system include the hamstring tendons (120) (on the back of the knee) which connect the hamstring muscles to the knee joint, patellar tendons (122) which connect the patella to the tibia and quadriceps tendons (124) which connect the quadriceps to the patella. Finally, there are the ligaments, posterior cruciate ligament (PCL) (130), medial collateral ligament (MCL) (128), lateral collateral ligament (LCL) (126), and anterior cruciate ligament (ACL) (132). The Anterior Cruciate Ligament (ACL) is the primary stabilizing knee ligament preventing the anterior translation of the tibia.

The ACL tears at approximately 2100 N of force. ACL tears can be broken down into two categories, of contact, and noncontact tears. These make up approximately 30% and 70% of ACL injuries respectively. Noncontact injuries can be further broken down into three categories of flexion/extension, internal/external rotation, and abduction/adduction, with two sub categories, of flexion/extension with rotation and abduction/adduction with rotation.

The ACL tears associated with flexion injuries are due to a phenomena known as Tibial Shear Force (TSF). When the leg is fully extended, there is a high amount of strain on the ACL from the tibia. The PCL, on the other hand, is relaxed. When the knee is flexed, the properties of the ACL and PCL switch. Once, the ACL tears, an event known as anterior tibial translation occurs. When the ACL tears, the tibia is released and moves forward. Flexion/extension related ACL injuries comprise approximately 37% of noncontact ACL injuries which is approximately 77,700 ACL injuries per year out of the total of approximately 300,000 each year.

Out of the approximately three hundred thousand ACL tears every year, approximately seventy eight thousand are flexion/extension related while around nineteen thousand are caused by abduction/adduction of the knee joint. Prior work has primarily been focused towards identification of the problem rather than preventive solutions. ACL injury repression programs mainly focus on building muscle memory associated with good form and development of muscles that support knee ligaments in an attempt to lower the probability of injury. One limitation of these programs occur in their implementation as only about 30% of coaches implement these programs correctly.

Implementational deficiencies associated with prior art approach to the problem may stem from limitation on how well a coach or trainer can feasibly monitor an athlete's movement for proper form. For example, it may be almost impossible for a coaching staff to observe an entire team resulting in a high likelihood that some participants form may escape observation. Furthermore, method of observing an athlete's form based on theories of kinesiology may be subjectively customized based on a particular coaching philosophy and/or experience. Therefore not only such methods becomes subjective in practice, there may also be limited by the ability and scope of human observation. There is a lack of quantitative methodology to determine good form conducive to minimizing ACL strain. Currently there are no systems or devices that can actively quantify ACL strain levels during dynamic movements and mitigate a potential injury or injury-prone form/posture/movement but accordingly alerting a user/athlete. As such, there exists a need for a precise system to actively quantify ACL strain levels and notify the athlete thus providing an opportunity to actively mitigate an injury-prone situation in real time.

One aspect of the present invention is directed toward a system and method for quantifying ACL strain and warning the user of strain levels that may contribute to flexion/extension related ACL injury. In accordance to one embodiment, this is accomplished by providing a wearable device that uses a Biofeedback Active Sensor System (BASS) to actively quantify the ACL strain during flexion modes, and subsequently warn the user of a high reading. In this way, a user/athlete is notified of their bad form and provided an opportunity to actively adjust their form to minimize probability of an ACL tear. Disclosed embodiments collect measurement reading of one or more sensors and convert the measurements into useable data. This data will then be used in equations to determine if an athlete is at risk of an ACL tear.

Some embodiments of the present invention utilize a TSF model for quantifying strain on the ACL. According to select embodiments, if TSF is higher than, for example, 1700 N of force, a warning is generated to notify the athlete of high TSF values generated as a result of the movement. Embodiments may utilize one or more sensors such as, for example, a flexion sensor, a ground reaction force sensors, or an accelerometer to support a TSF model based computation. In some embodiments, a value for the mass or weight of the athlete may be inputted into the system by the user

Two main operational scenarios for select embodiments of the disclosed invention include identification and mitigation. Identification is an active process that occurs in real time during dynamic movements. Mitigation is twofold; the first part is done actively during competition, while the second part may be performed afterwards with the aid of a trainer.

In accordance to one embodiment, an athlete will wear the BASS sleeve during practice and competition. During these events the athlete will perform as they normally do. The BASS will be continuously monitoring and calculating ACL strain. If there is a point when the ACL strain is too great, the BASS will alert the athlete through auditory, visual, tactical or textual means.

Mitigation scenario occurs when the athlete is alerted, for example by a beeping sound. The athlete can then make a mental note to have better form. Over the course of a dynamic event, the athlete may grow annoyed of the beeping and correct their form. After training, the athlete can view their TSF data. The athlete may then discuss the situation, with respect to their recorded TSF profile, with the coaching staff to implement ways to reduce the strain. The recorded TSF data may start off with many high strain levels at the beginning of a training session, as the session goes on, there should be a decreasing trend in the data due to the athlete's ability to neuromuscularly retrain themselves.

In order for a system to be put into place for mitigating ACL injuries, the physics of a knee flexion tear needs to first be understood. The knee flexion tear is caused by Tibial Shear Force (TSF). Tibial Shear Force may results from the force exerted by the momentum of the shank, the momentum of the foot, the earth's ground reaction forces, and the relative contributions of the Gastrocnemius muscle, the quadriceps muscle, and the hamstring muscle.

TSF=Fₛₕₐₙₖ+F_{Foot}+F_{gr}+F_{Muscle} (1)

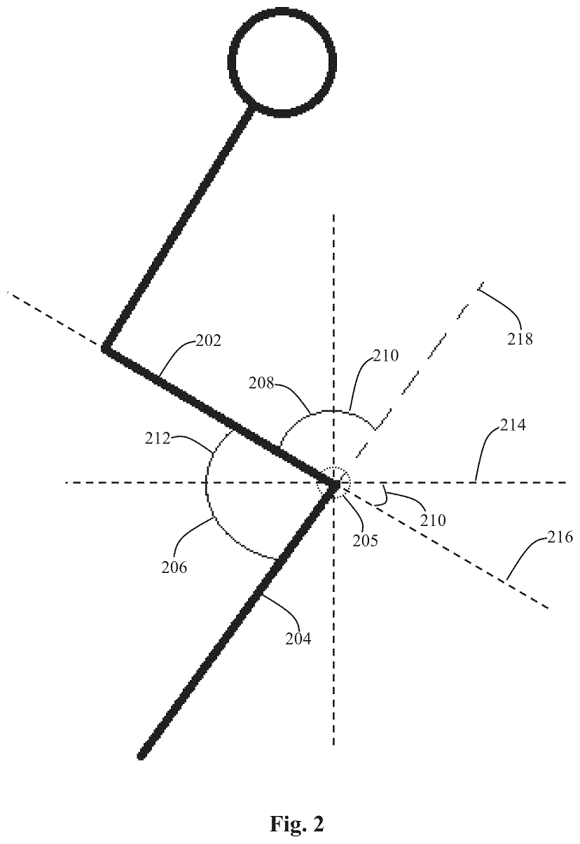

TSF=mₛ[aₛₓ cos(θₛ)−(a_{sy}+g)sin(θₛ)]+m_{f}[a_{fx} cos(θₛ)−(a_{fy}+g)sin(θₛ)]−F_{grx} cos θₛ+F_{gry} cos θₛ−ΣF_{gastro}_{ₓ}−ΣF_{Quad}_{ₓ}−ΣFₕₐₘ_{ₓ} (2)

Wherein parameters aₛₓ and a_{sy} represent the acceleration of the shank in the horizontal (x) and vertical (y) directions, respectively. Parameter θₛ represents the shank angle as shown by 210 in FIG. 2. Parameters a_{fx} and a_{fy} represent the acceleration of the foot in the horizontal (x) and vertical (y) directions, respectively. Parameters F_{grx} and F_{gry} represent the ground reaction force in the horizontal (x) and vertical (y) directions, respectively. Parameter F_{gastro}_{ₓ} represents the gastrocnemius muscle contribution to the TSF. Parameter F_{Quad}_{ₓ} represents the quadriceps muscle contribution to the TSF and parameter F_{Ham}_{ₓ} represents the hamstring muscle contribution to the TSF.

Examinations of the equations of motion as they relate to the knee joint system show the ground reaction force (F_{gr}) and muscle force (F_{Muscle}), specifically (F_{quads}_{ₓ}) and (Fₕₐₘ_{ₓ}) are the greatest TSF contributors when compared to the four main contributing factors shown in (1). Therefore (2) may be represented in a simplified form by (3)

TSF˜F_{gr}_{_{y}} sin θₛₕₐₙₖ−F_{gr}_{ₓ} cos θₛₕₐₙₖ−F_{quads}_{ₓ}−Fₕₐₘ_{ₓ} (3)

As illustrated in FIG. 1, the quadriceps muscle connects to the patella (knee bone) by a tendon which then connects to the tibia via a tendon. The relative force on the tibia is therefore relative to the position of the patella. The patella position changes due to knee flexion. Therefore the relative contribution to TSF from the quadriceps is due to flexion angle of the knee joint.

The muscle force contributions to TSF, therefore, have to be understood by the flexion angle, which is the relative angle between the femur and the tibia, as shown by 206 in FIG. 2.

FIG. 2 illustrates a simplified stick diagram 200 of a leg during a jump landing. Stick diagram 200 comprises a femur 202, a shank or the tibia 204 and a knee joint 205 disposed between the femur 202 and the shank 204. Flexion angles 206 and 208 of the knee joint 205 may be represented in terms of the shank angle (θₛₕₐₙₖ) 210 and the femur angle (θ_{femur}) 212 with respect to the main reference x-axis 214.

θ_{flex}_{ₐ}=90+θₛₕₐₙₖ−θ_{femur} (3)

θ_{flex}_{_{b}}=90−θₛₕₐₙₖ+θ_{femur} (4)

As illustrated earlier in FIG. 1 The femur is the thigh bone which supports the muscles involved in the knee joint system. The patella, also known as the knee cap, allows for joint movement. The tibia and fibula make up the shank (lower part of the leg). Leg muscles comprising the quadriceps, hamstrings and gastrocnemius may all act to mitigate the strain on the ACL. The quadriceps applies the most force, followed by the hamstrings, and then the gastrocnemius. The ligaments comprising of PCL, MCL, LCL and ACL, act as stabilizers of the knee which prevent the tibia (shank) from sliding forward and out from under the femur.

In order to evaluate TSF contributions from the leg muscles (quadriceps, hamstrings and gastrocnemius) represented collectively as F_{muscle} in (1), the force contribution of each muscle type is determined. The muscle force contributions to TSF have to be understood by the flexion angle, which is the relative angle between the femur and the tibia, as mentioned earlier

The force contribution of the quadriceps muscle (F_{Quad}_{ₓ}) exerted along the shank's reference axis 216 is shown in (5)

F^{s}_{quad}_{ₓ}=F_{quad}*sin(θₜ₂ₜ) (5)

Wherein the angle between the patellar tendon 122 and tibia (shank) bone 108 is expressed as θₜ₂ₜ such that:

when θ_{flex}_{_{b}}=180,θ_{flex}_{ₐ}=0 then θₜ₂ₜ=22.2°

when θ_{flex}_{_{b}}=70,θ_{flex}_{ₐ}=110 then θₜ₂ₜ=−4.03°

Therefore, assuming that the patella 106 can pull in the negative direction, θₜ₂ₜ and F^{s}_{quad}_{ₓ} are expressed as equations (6) and (7), respectively.

θₜ₂ₜ=(−0.238)(θ_{flex}_{ₐ})+22.2 (6)

F^{S}_{quad}_{ₓ}=F_{quad}*sin((−0.238)*(180−0_{flex}_{_{b}})+22.2° (7)

The relative force on the tibia is therefore relative to the position of the patella, which changes due to knee flexion (θ_{flex}_{_{b}}). Therefore, as illustrated by (7) the relative contribution to TSF from the quadriceps is due to the flexion angle.

The hamstring muscle contributes to TSF by pulling the tibia in the opposite direction as the quadriceps. In this way the hamstring is the largest muscular mitigator of TSF. The hamstring contribution is directly related to the flexion angle. The force contribution of the hamstring muscle (F_{hamstring}_{ₓ}) exerted along the shank's reference axis 216, as shown in FIG. 2, is may be expressed by (8)

F^{s}_{hamstring}_{ₓ}=|F_{hamstring}*cos(|90−θ_{flex}_{_{b}}∥ (8)

The force contribution of the gastrocnemius muscle (F_{gastrocnemius}_{ₓ}) exerted along the shank's reference axis 216 is shown in (9)

F^{s}_{gastrocnemius}_{ₓ}=F^{s}_{gx}=F_{g}*sin(θ_{g}) (9)

Wherein the angle of the gastrocnemius muscle relative to the tibia (θ_{g}) may be expressed as a function of the flexion angle θ_{flex}_{_{b}} and the length of the gastrocnemius (l_{g}) as shown in (10)

$${θ}_{gastrocnemius} = {θ}_{g} = {sin}^{- 1} [ \frac{d * sin ( {θ}_{f l e {x}_{b}} )}{{l}_{g}} ] \qquad ( 10 )$$

With l_{g} expressed as:

$${l}_{g} = \sqrt{{( d )}^{2} + {( {l}_{tibia} )}^{2} - 2 * ( d ) * ( {l}_{tibia} ) cos ( {θ}_{{flex}_{b}} )} \qquad ( 11 )$$

Wherein d represents the distance from the gastrocnemius muscle connection point on the femur to the knee and may be approximately 3 centimeters.

The gastrocnemius muscle (calf) contributes to TSF by connecting to the lower part of the femur. In this way, similar to the hamstring muscle, it also creates a TSF force in the opposite direction of the quadriceps. It mitigates TSF but not in the magnitude of the hamstring muscles.

Substituting (11) and (10) into (9), the gastrocnemius muscle force contribution (F_{gastrocnemius}_{ₓ}) exerted along the shank's reference axis 216 may be expressed as shown in (12)

$${F}_{gx}^{s} = {F}_{g} * sin ( {sin}^{- 1} ( \frac{d * sin ( {θ}_{{flex}_{b}} )}{\sqrt{\begin{matrix}{( d )}^{2} + {( {l}_{tibia} )}^{2} - \\ 2 * ( d ) * ( {l}_{tibia} ) cos ( {θ}_{{flex}_{b}} )\end{matrix}}} ) \qquad ( 12 )$$

The ground reaction force (F_{gr}) contribution to TSF is moderated by the body's dissipation of the ground impact forces as determined by Newton's laws. The Ground reaction forces counteract the body's center of mass and momentum which is based on the athletes predetermined neuromuscular response to their landing goals. It can be thought of as a smart spring that an athlete's form would have to be analyzed to derive.

Ground reaction force (F_{gr}), as shown in (13), is comprised of a vertical component (F_{gr}_{_{y}}) and a horizontal component (F_{gr}_{ₓ}).

F_{gr}={square root over (F_{gr}_{ₓ}²+F_{gr}_{_{y}}²)} (13)

The vertical component (F_{gr}_{_{y}}) of the ground reaction force expressed in terms of the mass of the falling body (M_{b}), height of the drop (h), acceleration due to gravity (g) and impact distance in the vertical direction (ΔY_{impact distance}) is shown in (14).

$${F}_{{gr}_{y}} = \frac{{M}_{b} * h * g}{Δ {Y}_{impact distance}} \qquad ( 14 )$$

The horizontal component (F_{gr}_{ₓ}) of the ground reaction force in terms of the mass of the falling body (M_{b}), height of the drop (h), acceleration due to gravity (g) and the impact distance in the horizontal direction (Dₓ) as shown in (15).

$${F}_{{gr}_{x}} = \frac{{M}_{b} * {D}_{x}}{\sqrt{\frac{2 * h}{g}}} \qquad ( 15 )$$

Substituting (14) and (15) into (13) results in the total ground reaction force as expressed in (16)

$${F}_{gr} = \sqrt{{[ \frac{{M}_{b} * h * g}{Δ {Y}_{impact distance}} ]}^{2} + {[ \frac{{M}_{b} * {D}_{x}}{\sqrt{\frac{2 * h}{g}}} ]}^{2}} \qquad ( 16 )$$

In order for the body to avoid slipping along the horizontal landing upon impact, the horizontal component (F_{gr}_{ₓ}) of the ground reaction force must be greater than the landing surface frictional forces (F_{friction}_{_{impact}}) experienced at the instance of impact (17). This relation is illustrated in (18).

$$\begin{matrix}{F}_{{fτrction}_{impact}} = {μ}_{s} * \frac{{M}_{b o d y} * h * g}{Δ {Y}_{impact distance}} & ( 17 ) \\ \frac{{M}_{b} * {D}_{x}}{\sqrt{\frac{2 * h}{g}}} > {μ}_{s} * \frac{{M}_{b o d y} * h * g}{Δ {Y}_{impact distance}} & ( 18 )\end{matrix}$$

Flexion/Extension injuries usually occur due to low flexion angles and make up 37% of total ACL tears. This is because of a phenomena known as quad dominance where the quadriceps, instead of mitigating an ACL tear, actually apply strain to the ACL that may result in an ACL tear.

This happens because of the way all the components are linked in this system. As was illustrated in FIG. 1, the quadriceps muscle is attached to the quadriceps tendon which is connected to the patella which is connected to the patellar tendon which is finally connected to the shank, specifically the tibia. Therefore form and position of the body while landing, key factors. At high flexion angle of the knee (angle between the femur and the shin), for example, when in a low squat, the quadriceps is pulling the shank backwards, and reducing strain on the ACL. When straight legged, the quadriceps chain is actually pulls the shank forward. This produces strain on the ACL and may cause it to break. If and when that happens, a phenomena known as anterior tibial translation occurs. Anterior tibial translation is the shifting of the tibia forwards, out in front of the femur. This distance is about 16.7 mm but that varies from person to person.

Therefore, the flexion angle 206, illustrated in FIG. 2 needs to be below a certain threshold. If there is too little flexion the quadriceps pulls the shank forward and the hamstring and calf muscles cannot counteract it, causing the shank to slide out from under the femur

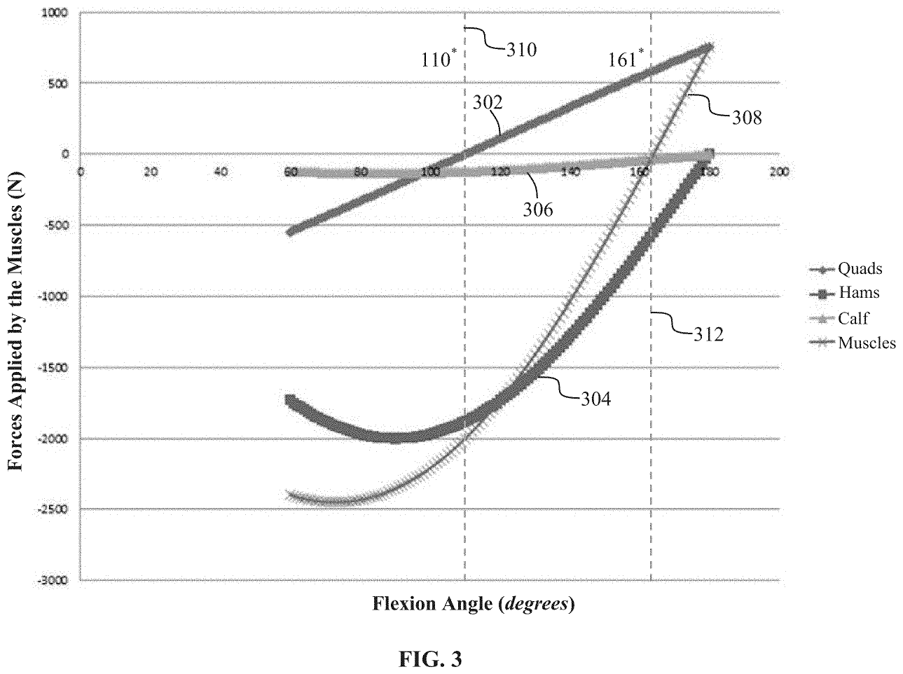

A sensitivity analysis of the TSF model, to analyze the contributions of leg muscle activation to the overall TSF, is carried out by varying the flexion angle while holding all other parameters at their average value. The result of the sensitivity analysis is shown in FIG. 3.

FIG. 3 illustrate a profile of quadriceps muscle force contribution (trace 302), hamstring muscle force contribution (trace 304), calf muscle force contribution (trace 306) and overall leg muscle force contributions (trace 308) to the TSF as a function of the flexion angle. As shown by the marker line 310 at approximately 110 degree the quadriceps muscles starts to contribute positively to the TSF, counteracting the restorative contribution of the hamstring and calf muscle. At approximately 160 degrees, denoted by marker line 312, the quadriceps muscle force dominates the hamstring and gastrocnemius muscle in the TSF reference and therefore contributes more overall force to TSF. This verifies the concept of “Quad-dominance” which refers to the tendency to absorb ground reaction forces with flexion angles lower than 20 degrees.

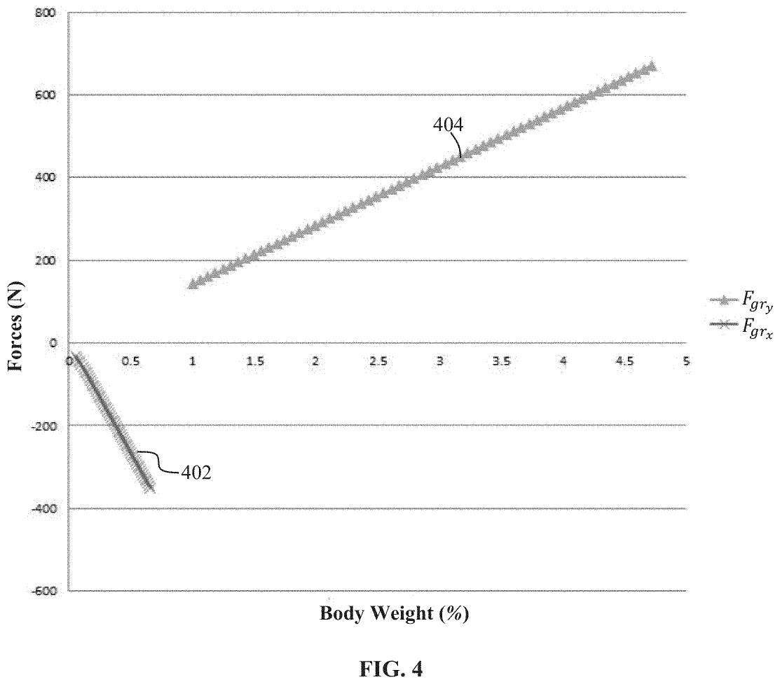

FIG. 4 illustrates the results of analyzing the contribution of the vertical and the horizontal component of ground reaction force to TSF. The analysis is conducted by keeping a constant shank angle and computing the effects to the TSF by varying the body weight. As illustrated by trace 402 the horizontal component (F_{gr}_{ₓ}) of the ground reaction force actually dissipates overall TSF while the vertical component (F_{gr}_{_{y}}) of the ground reaction force (trace 404) contributes linearly to a maximum of 700 N of force.

Therefore, based on sensitivity analysis preformed on the TSF equation, the ground reaction force in the vertical direction (F_{gr}_{_{y}}) and flexion angle θ_{flex}_{_{b}} may be designated as two largest contributing factors to the overall TSF which approximates the ACL strain. Therefore TSF, hence the ACL strain may be approximated as a function of knee flexion and vertical ground reaction force, or instantaneous momentum, upon impact

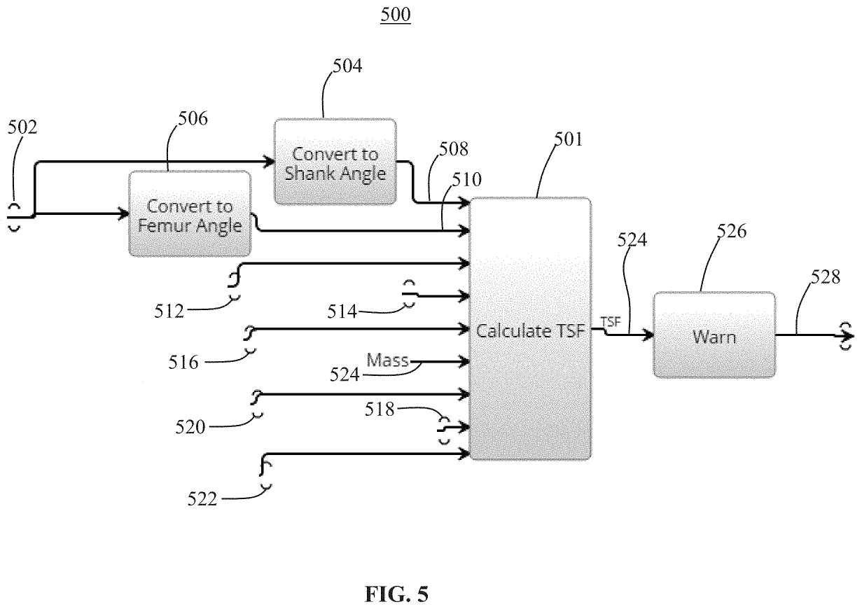

FIG. 5 illustrates a exemplary ACL injury prevention system operationally based on the TSF model shown in (2). System 500 does not incorporate simplifications associated with the TSF model sensitivity analysis discussed above. Therefore to actively monitor all contributing factors to TSF as expressed by TSF equation (2), system 500 utilizes measurement readouts from multiple sensors, as inputs into a processing unit 501, for quantifying all the components of the TSF equation (2). System 500 deploys 7 sensors comprising, sensor 502 for measuring flexion angle of the knee, wherein sensor output is then fed through module 504 and 506 in order to convert sensor output to useful inputs for shank angle 508 and femur angle 510, respectively. System 500 further comprises a jump distance (Dₓ) sensor 512, a jump height (h) sensor 514, foot acceleration sensors 516 and 518 for measuring the vertical foot acceleration component (a_{fy}) and horizontal foot acceleration component (a_{fx}), respectively. Additionally, system 500 includes shank acceleration sensors 520 and 522 for measuring the vertical shank acceleration component (a_{sy}) and horizontal shank acceleration component (aₛₓ), respectively. System 500 also includes an input 524 for receiving user entered parameters i.e., user's mass or weight, as required by TSF equation in (2). The system 500 will then route all input parameters along with sensor readings to a processing unit 501 wherein the data may be actively run against a TSF algorithm in order to calculate the TSF value 527. The processor 501 will determine if the calculated TSF value 527 is above the acceptable ACL safety threshold limit. If the threshold is crossed, the system activates the warning system 526 to generate one or more types of warning signals 528 thus actively notifying a user of their bad form.

According to embodiment of the invention, horizontal and vertical components of acceleration for either the shank or the foot maybe measured using a single triaxial acceleration sensor that measures the acceleration along both the X and Y axis.

According to an embodiment, system 500 may be mounted onto a brace, such as, for example, a knee brace that fits around the knee joint.

Based on analysis of the major TSF contributing and mitigating factors described earlier and illustrated in FIG. 3 and FIG. 4 one embodiment of the invention disclose a system comprising a sensor for measuring vertical ground reaction force and a sensor for measuring a flexion angle of the knee. The system may then generate one or more warning signals when sensor outputs become indicative of a high TSF value associated with detrimental levels of strain on the ACL.

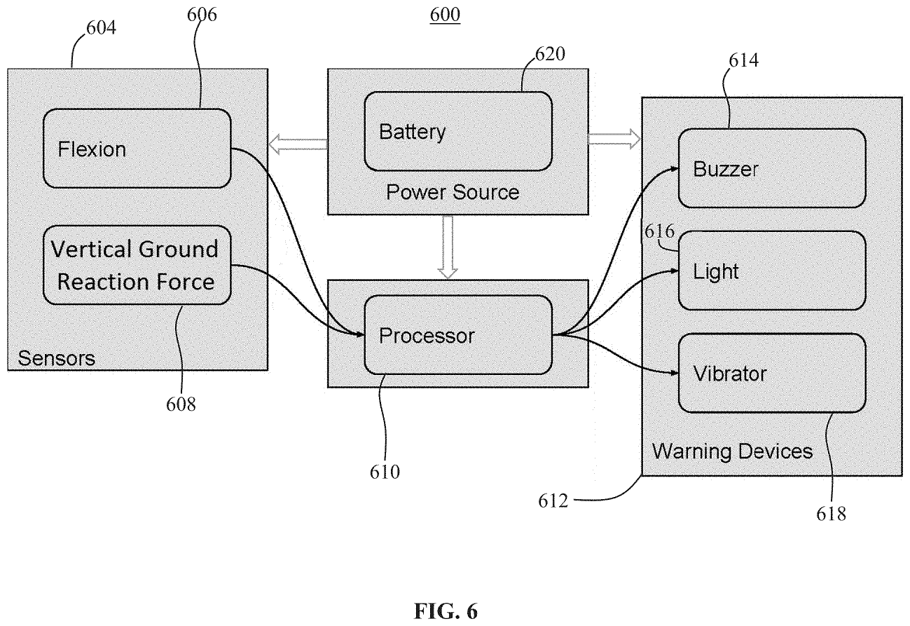

FIG. 6 illustrates an operational overview of a system 600 implemented based on sensitivity analysis of TSF as a function of knee flexion angles and vertical ground reaction force.

System 600 comprises a sensing module 604 having one or more sensing units comprising a flexion angle sensor 606 and a vertical ground reaction force sensor 608. A processing unit 610 receives the output of the flexion angle sensor 606 and vertical ground reaction force sensor 608 and activates a warning system 612 when sensor measurements are deemed to correspond to high levels of TSF which would indicate a detrimental amount of ACL strain. The warning system may comprise one or more auditory alert devices such as buzzer 614 and/or one or more visual alert devices, such as light 616 and/or one or more tactical alert devices, such as vibrator 618. The system is responsive to sensor readout combinations (i.e., flexion angle and ground reaction force value pairs) associated with high TSF levels. A power source, such as a battery unit 620, may be used for powering the sensing, processing and warning modules.

In one embodiment, flexion angle and ground reaction force value combinations that correspond to high TSF levels may be programmed into the processing unit 610. When the processing unit receives sensor data from 606 and 608, that match or exceed stored value pairs associated with unsafe TSF levels, it will generate one or more warning signals to alert the user/athlete of high level of ACL strain and to prompt the user/athlete to make necessary adjustments in order to mitigate a potential ACL injury.

High TSF may be defined as a buffered level below ACL tear threshold so the system may prevent injury by alerting a user before an ACL injury may occur. In accordance to one embodiment of the invention a TSF threshold of 1800 N in conjunction with knee flexion of less than 21 degrees may be selected as a warning threshold.

Some embodiments of the invention may comprise one or more pressure sensors for directly measuring the vertical ground reaction force. This may be implemented through placing one or more pressure sensors in the insoles of an athlete's shoes as part of an ACL injury prevention system. The total vertical ground reaction force is found through totaling the forces measured by one or more pressure sensor. Other embodiments may comprise an accelerometer for determining a vertical ground reaction force. Accelerometers records the acceleration along the y-axis of the knee system which may then be used to determine an instantaneous momentum upon ground impact as an indication of a vertical ground reaction at the knee joint. The acceleration data generated by the accelerometer may also be multiplied by weight of a user to calculate an impact force upon landing.

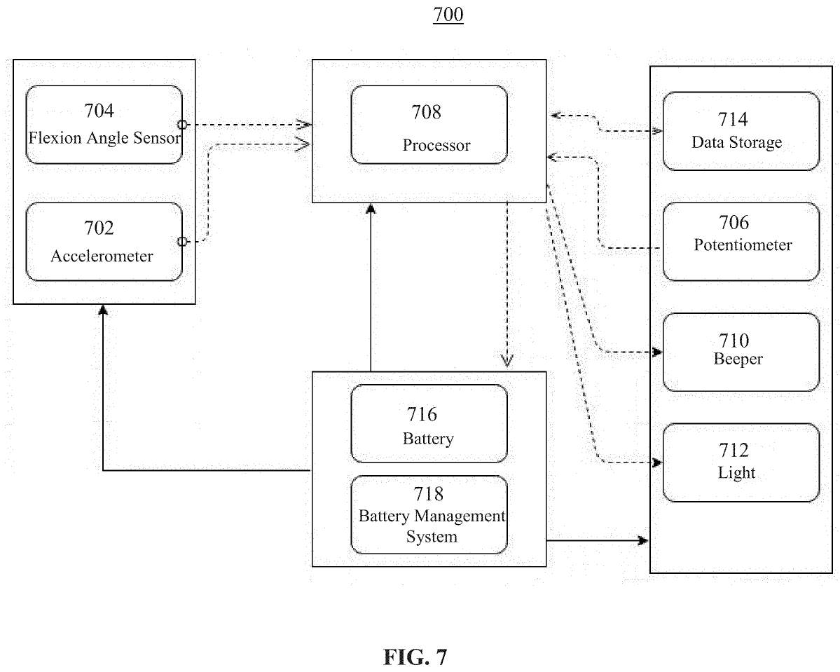

FIG. 7 illustrates an exemplary knee joint injury prevention system 700 that incorporates an accelerometer 702 and flexion angle sensor 704, both of which are electrically coupled to a processing unit 708. Processing unit 708 may utilize the output of the accelerometer 702 for quantifying an impact force transmitted to an anatomical joint such as the knee joint upon landing. The impact force may be caused by vertical ground reaction force at the knee joint and may thus be quantified by determining an instantaneous momentum of the knee joint system upon landing (ground impact). Accelerometer 702, therefore, actively sends acceleration data to the processing unit 708. According to one embodiment, processing unit 708 may use Newton's equations to convert the output of the accelerometer into vertical ground reaction force at the knee joint.

Similarly, the flexion sensor 704 actively sends output data corresponding to knee flexion angles to processing unit 708. Processing unit 708 receives sensor readings from 702 and 704 and when a threshold is met, for example, when the processing unit 708 reads high knee accelerations in the vertical (Y) direction in conjunction with a flexion angle that does not exceed a threshold limit within a reasonable amount of time, the processing unit 708 initiates an auditory and/or visual warning sequence to the user by activating beeper 710 and/or light 712.

A battery 716 and battery management system 718 may be utilized to power the sensing, processing, warning and peripheral units of system 700. System 700 may further comprise a data storage unit 714 for recording sensor outputs or relevant computations of the processing unit in order to facilitate post dynamic activity review by a user. Processing unit 708 may further be electrically coupled to a potentiometer 706 for receiving user entered parameters, such as user weight or mass that may be used for one or more computations of the processing unit 708. The beeper 710 and light 712 may act as a dual warning system. For example, beeper 710 may alert the athlete of high ACL strain while light 712 may alerts the coach.

In accordance to one embodiment, measurement of joint flexion angle may be carried out using variable resistor sensor such as, for example, flex sensors. Flex sensors comprise bendable angle measuring devices having an electrical resistance which increases as the body of the flex sensors is bent, thus enabling a measurement of flexion angle based on a change in resistance of the device. Flex sensor outputs maybe used in a filter, such as an extended kalman filter (EKM) to minimize the error of flexion angle reading.

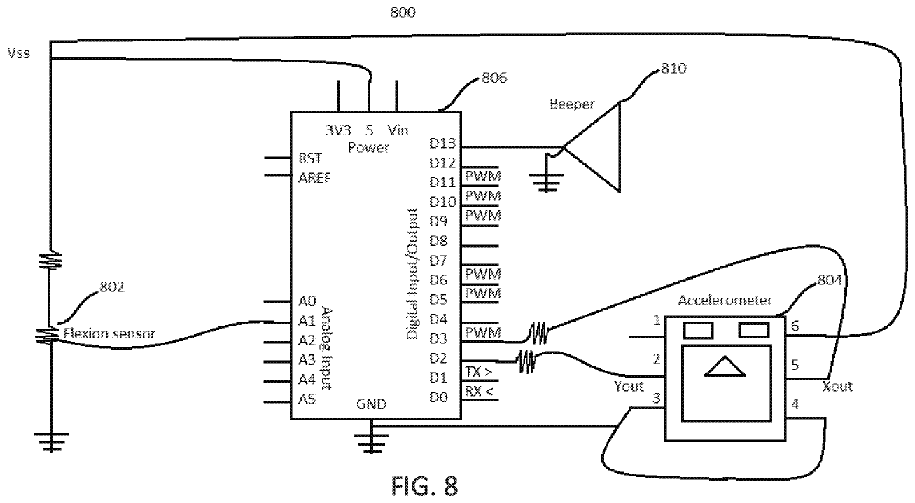

FIG. 8 illustrate an exemplary system 800 comprising a variable resistor sensor, such as flexion sensor 802, for the measurement of joint flexion angle, an accelerometer 804 for the measurement of a vertical acceleration of the joint system that may be used to quantify vertical ground reaction force at the joint upon impact. System 800 may further comprise a processor 806 for receiving output of 802 and 804 and activating one or more alarm units, i.e., beeper 810, upon recording sensor (802, 804) readouts commensurate with unsafe levels of TSF that may be conducive to ACL injury.

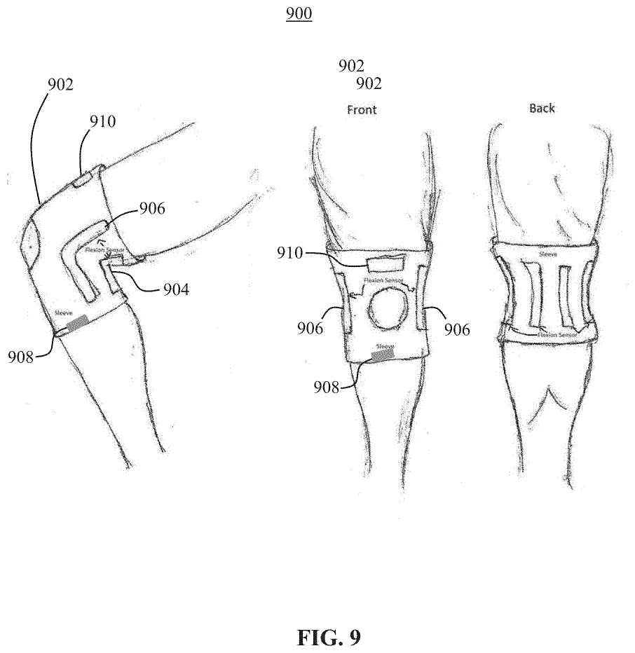

In accordance to an embodiment of the invention, all components of a knee joint injury prevention system as illustrated in FIGS. 5, 6, 7 and 8 maybe attached to a wearable system, such as a joint sleeve or brace to facilitate monitoring of the joint environment. An exemplary wearable knee joint prevention system, in accordance to one embodiment of the present invention is illustrated in FIG. 9.

The exemplary wearable knee joint injury prevention system 900 comprises a knee sleeve/brace 902 acting as a joint brace, in addition to an attachment surface for various components of the knee injury prevention system. Since the knee is a hinge with a mechanical advantager called the patella. The patella increases the mechanical advantage of the quadriceps muscles on the tibia (lower leg) but also can produce negative effects to the joint under high stress. This high stress is the force that the body must produce to keep standing. Therefore, that force will be sensed by a combination of an accelerometer at the knee to produce instantaneous ground reaction force. And an addable pressure sensor at the bottom of the foot in the form of a special sock. The angle of the knee shall be calculated with four flexion sensors, 906 and 904, surrounding the knee. Four is needed because the knee plus patella system needs precise measurements and four sensors will reduce the error in measurement. The rotation at the knee will be sensed by two gyroscopes 910 at the knee and at the ankle. The first gyroscope will compare its angle with the second gyroscope at the ankle to tell the relative angle between the foot and knee and then calculate it with the readings at the knee accelerometer and flexion sensor to see if there is excessive torque at the knee. Wearable system 900 comprises two flexion sensors, for measuring flexion angle of the knee, disposed on the knee sleeve/brace at 904 and 906. Wearable system 900 further comprises an accelerometer disposed on the knee sleeve/brace 902 at 908 and used for measuring a downwards accelerations of the knee joint, and a processing unit disposed at 910 for processing the output data from the accelerometer and flexion sensor and for generating feedback to a user with regard to an ACL injury prone form, posture or movement. In accordance to an embodiment, warning and/or feedback generation system is activated in response to sensor data corresponding to unsafe levels of TSF, as determined by an algorithm running on the processor. Unsafe levels of TSF may be identified by a likelihood of ACL injury occurrence.

According to one embodiment of the present invention the disclosed method and system trains an athlete to keep good form by using an alert system which alerts the user of improper form, based on output data from one or more tactically placed sensors.

One embodiment of the disclosed system is directed at dynamically identifying and warning a user of high levels of TSF on a knee joint system. The embodiment may utilize an internal algorithm running on an internal processor to quantify, based on collected sensor data, the TSF experienced by an anatomical joint, and utilize the result as an approximate indicator of ACL strain.

One embodiment of the disclosed system wireless interface, such as, for example Bluetooth, for communicating one or more sensor outputs to a specialized application running on a portable computer such as, for example, an electronic tablet, smart phone and/or smart watch.

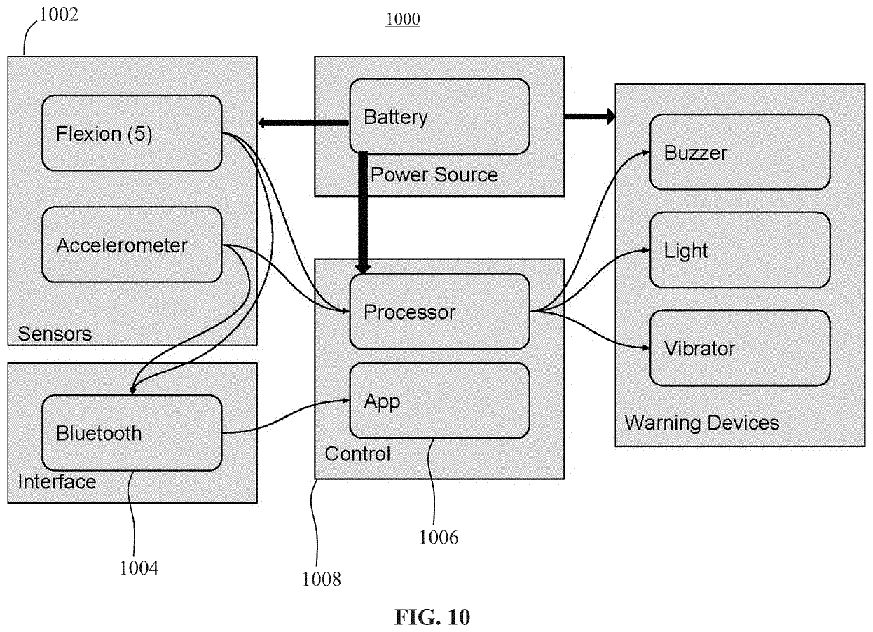

FIG. 10 illustrates an anatomical joint injury prevention system 1000, in accordance to one embodiment of the invention. In system 1000, data measurements generated by sensing module 1002 are made available, wirelessly through a Bluetooth interface 1004, to a specialized application 1006 running on a portable processing unit 1008. The specialized application 1006 may then carry out the necessary processing of sensor outputs, utilizing processing and memory resources of the portable host computer 1008 such as, for example, a smart phone running the application, in order to determine unsafe TSF levels associated with ACL injury. The output of the specialized application 1006 may comprise one or more warning signals and/or feedback information reported to the user.

The specialized application 1006 may be user configurable to enable a user to choose different training methods, in accordance to one embodiment of the invention. To do this a user may use a smart phone that runs a BASS smart phone application. The smart phone application may have different training modes loaded that have instructions and videos on how to do perform various movements to minimize risk of ACL injury. The specialized application running, for example, on a smart phone, may utilizes sensor data that may be received via a wireless interface such as Bluetooth. The specialized application may then utilize computational power and memory resources of the portable host computer, such as a smart phone running the application, to generate feedback comprising of accurate and persistent form analysis, one or more user-tailored workout regimen and/or a user progress report.

Having described the many embodiments of the present invention in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

For example the disclosed method and system for preventing knee joint injuries may be employed as part of an overall NeuroTraining system. The NeuroTraining system may be comprised of several individual components or modules all of which govern good form training in different athletic movements by offering different training modes.

In an exemplary embodiment, the disclosed method and system for preventing knee joint injuries may be implemented within NeuroTraning knee sleeve that monitors athletic movement effects on the knee. By performing the provided workout regimens and following the adaptive personalized workout routine, a user will lower their probability of ligament tear (including the ACL tear) cartilage wear (preventing osteoporosis in the process) and increase athletic performance by correcting a phenomenon called quadricep dominance which has been found to effect women 3 times more than their male counterparts. The design of the Neurotraining System relies on sensors that collect angles and forces at certain failure points in the body. There are six failure main points that the system may include, knee, shoulder, back, elbow, ankle, hip, and spine.

The NeuroTraining system may also comprise a NeuroTraining shoulder sleeve module that monitors the complex shoulder joint. The shoulder is used in all upper body activities and therefore has to be very flexible and versatile. With all this comes weakness due to the potentiality of muscular imbalances and the effects thereof. The shoulder joint is a rotating ball joint and therefore needs the angles of flexion in four axis. This is derived by four flexion sensors surrounding the shoulder sewn into a shirt. The angle of rotation of the humorous (arm bone) within the shoulder ball socket will be derived by a gyroscope at the elbow and at the top of the shoulder. The force at the shoulder will be calculated from the modes and the assumptions there of. An example would be baseball throw mode would assume a baseball at the end of the lever arm there for the force at the shoulder would be a function of the angle of the elbow and the frequency of rotation of the shoulder holding a 1 lb baseball and the acceleration. Another example would be push up mode. This would use the angle of the humorous compared to the shoulder and a function of the users body weight. A bench press mode would use the same algorithm but with different weight input. By using the NeuroTraining shoulder sleeve, a user will lower their probability of injury from different activities like weight lifting, baseball, golf, tennis and boxing. Increased performance will occur as good training form is tailored to activate the most efficient muscles and therefore training them to be stronger. Muscle imbalances are also decreased in this manner.

The NeuroTraining system may also comprise a Neurotraining back brace module that helps correct a user from performing movements that can strain the spine or large muscles supporting the lumbar system. This back brace is not only meant for athletic training like football and other power sports, but also for workers that regularly lift objects from the ground and overhead. The spine is a chain of rotating columns. The angle of spinal bend will be sensed by a flexion sensor along the spine. The angle of spinal twist will be sensed by a gyroscope at the top and bottom of the spine. The force will be from the mode and the difference of readings in the two gyroscopes over time. By wearing this lightweight breathable device while working, a user will then be alerted by their bad form and either correct it or stop the potential debilitating movement that they were performing, thereby saving against the time and cost of work loss due to injury. Athletic performance will be increased by making sure the correct muscles are being trained.

The NeuroTraining system may also comprise a Neurotraining hip module that helps correct a user from performing movements that can strain the hips and large muscles supporting it. The hip is a rotating ball joint. The angle will be sensed by flexion sensors at 4 points that give the angles of the leg to the hips. The force will be calculated the same way as at the knee with a combination of the accelerometer at the knee and the pressure sensor at the foot. By wearing device while working, a user will then be alerted by their bad form and either correct it or stop the potential debilitating movement that they were performing, thereby saving against the time and cost of work loss due to injury. Athletic performance will be increased by making sure the correct muscles are being trained.

The NeuroTraining system may also comprise a Neurotraining elbow module that helps correct a user from performing movements that can strain the elbows and muscles supporting it. The elbow is a hinge joint with no mechanical advantage like the patella for the knee. The angles at the elbow in 1 axis. Two flexion sensors on each side of the elbow are needed to determine that. The force at the elbow will be calculated by a combination of the mode (algorithm) and the flexion sensors and gyroscope system between the elbow and shoulder which would give elbow rotation. Flexion force would be the delta of elbow flexion over time. By wearing device while working, a user will then be alerted by their bad form and either correct it or stop the potential debilitating movement that they were performing, thereby saving against the time and cost of work loss due to injury. Athletic performance will be increased by making sure the correct muscles are being trained.

The NeuroTraining system may also comprise a Neurotraining ankle sleeve senses both abducting flexion, rotational flexion and ground reaction force. The ankle is a hinge type of joint. The angle of the ankle will be sensed at the top of the foot with a flexion sensor. The force will be collected with an accelerometer at the ankle. The rotational angle will be sensed by a gyroscope at the ankle. Foot placement during exercise contributes to knee injuries so by training proper foot placement during activity, the rotational knee injury that has plagued many athletes to do ACL tears and the effects on careers, will fade away. The heals impact to the ground that contributes to knee degradation and hip bursitis will also be lowered through using the knee and ankle training regimen. Ankle injuries will be lowered through the use of this divice and the knee sleeve because ankle strain correlates with knee flexion.

They NeuroTraining system may be broken up into two subcategories, an upper body system and a lower body system. The upper body system shall have an elbow sleeve and shirt. The lower body system shall have a knee sensor and an ankle sensor.

The elbow sleeve shall have 2 flexion sensors to tell the angle of the humorous compared to the radius and 1 gyroscope to tell the rotation of the humorous compared to the shoulder and to give the torque applied on the elbow when used in tandem with the flexion sensors. It will also have 1 battery to power the blue tooth interface and 1 blue tooth interface to interface with the processor in the shirt and therefore the app in the phone.

The Sensor shirt shall have 9 flexion sensors to give the angles of flexion for both shoulders and the bend angle of the spine and 4 gyroscopes, 2 at the top of the shoulder to be used with the elbow gyroscopes to tell the rotation of the humorous to the shoulder socket and 2 at the top and bottom of the back to tell the angle or rotation of the spinal column. It will also have 1 blue tooth interface to collect data from the elbow sleeves and to interface with the phone app, 1 battery to power the device, 1 warning system to sound, vibrate or light a waning of bad form to the user based on the exercise mode, and 1 processor to enable the algorithm to trigger the warning system and to translate messages to the phone app.

The knee sensor shall have one or more flexion sensors to give precise knee flexion angles, a gyroscope to be used with the ankle gyrsoscope to read tibia rotation in comparison to the knee, and a accelerometer to give knee vector and instantaneous ground reaction force. It will also have a battery to power the device, a blue tooth device to collect data from the ankle sleeve to communicate with the phone app, a warning system to sound vibrate or light to warn a user of bad form, and 1 processor to house the training mode and to trigger the warning system and to send data to the Bluetooth device.

The ankle sensor will have 1 flexion sensor to read the flexion of the ankle and 1 gyroscope to be used with the knee gyroscope to tell the rotation angle of the tibia to the knee joint. It will also have a bluetooth interface to interface with the knee sleeve, a battery to power the device, and a pressure sensor array to give pressure readings that speak to ground reaction force.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

For example, a wearable device that mitigates the probability of ACL tear by warning against bad form is disclosed. In an environment wherein there are 300,000 ACL injuries a year within the United States alone, an ACL can be torn with excessive force of around 2100 newtons. There are 5 ways to tear an ACL 37% of ACL injuries happen because the quadriceps muscle pull the tibia forward past the femur when the knee absorbs high ground reaction force and the knee is in a low flexion angle.

As stated above, there are 300,000 ACL injuries a year within the united states alone, An ACl can be torn with excessive force of around 2100 newtons. There are 5 ways to tear an ACL 37% of ACL injuries happen because the quadriceps muscle pull the tibia forward past the femur when the knee absorbs high ground reaction force and the knee is in a low flexion angle. The invention claimed here solves this problem.

Since these tears happen when an athlete absorbs ground reaction force with low flexion angles, or bad form, this device will warn the athlete by sound that they have landed with high force at low flexion angles, Over time the user learns good form by trying to keep the device from beeping. Landing with good form will lower ACl strain and therefore lower the probability of tearing an ACL

The claimed invention differs from what currently exists. This device is the first of its kind by using an alert system to train an athlete to keep good form. This device can be used by a lone individual or an instructor/team system to achieve results.

This invention is an improvement on what currently exists. This device is the first of its kind by using an alert system to train an athlete to keep good form. This device can be used by a lone individual or an instructor/team system to achieve results.

In a disclosed embodiment, the invention may comprise:

1. MicroProcessor

2. Wearable knee sleeve

3. Accelerometer

4. Flexion Sensor

5. Beeper

The (3) Accelerometer uses newtons equations to read ground reaction force into the (1) Microprocessor. The (4) Flexion sensor reads the knees flexion angle into the (1) Microprocessor. The (1) microprocessor takes the readings from (3) and {4) and when a threshold is met, the (1) microprocessor sends a signal to the (5) beeper. (1), (3), (4) and (5) are all attached to the knee sleeve in ergonomic areas.

The (3) accelerometer actively sends acceleration data to the (1) microprocessor. The (4) flexion sensor actively sends knee flexion angles to the (1) microprocessor. When the (1) microprocessor reads high knee accelerations in the Y direction and that the knee never flexes past a certain angle within a reasonable amount of time, the {1) microprocessor sends a signal to the (5) beeper for it to beep. All components are attached to the (2) wearable knee sleeve so they can monitor the knee environment.

If the (3) accelerator reads a high enough acceleration, then the (1) microprocessor checks the (4) flexion sensor to see if knee flexion ever goes past a certain threshold. If the (4) flexion sensor doesn't go past a certain angle, then the (5) beeper beeps,

A person would sew a pocket for the (1) microprocessor to be attached to the bottom left of the (2) wearable knee sleeve. Then they would sew a long thin pocket on the front of the knee sleeve to store the (4) flexion sensor. The person would then sew a pocket for an (3) accelerometer to be attached next to the (1) microprocessor. The person would sew a pocket for the (5) beeper to be on above the (1) and (3). The (4) flexion sensor would be wired to the (1) microprocessor. The (3) Accelerometer may be wired to the (1) microprocessor. The (5) beeper may be wired to the microprocessor.

The above elements of the (1) microprocessor, (2) wearable knee sleeve, (3) accelerometer, (4) flexion sensor are necessary to tell if a user has bad form at an athletic movement in a flexion move. The (5) beeper is the device used to warn the user of bad form and to give them the opportunity to correct their form. The (5) beeper could be interchanged with a vibration mechanism, a light mechanism, or a memory storage mechanism to collect bad form data for later analysis.

If the (4) flexion sensor was attached to the rear or the side of the knee sleeve, then the device would still read flexion angle and still work. The locations of the other components are arbitrary and wont effect the devices effectiveness. The (3) accelerometer will have to be positioned with the y reader pointing towards the sky for the device to work.

A lone user would wear the knee sleeve and preform a jump exercise. If the user lands a high enough jump with lower flexion angle then the device would beep. When a user hears the beep they would know they performed the jump with bad form and therefore high ACL strain. By minimizing the amount of beeps they get, they would minimize overall ACl strain and maximize their protection from injury.

In a team training environment, the team would perform jump exercises and a trainer would be able to actively focus on correcting individuals form but also able to hear the device warning of other users bad form. In this way a trainer could have total knowledge of a team's good or bad form while performing jump exercises.

Additionally: this inventions algorithm could be adjusted to read poor long distance running form. By shortening the allowable acceleration threshold, a user would be warned of high Y ground reaction force which would lead to an injury caused by the repetitive nature of running.

Thus, a wearable device is provided by a disclosed embodiment that mitigates the probability of ACL tear by warning against bad form is disclosed.

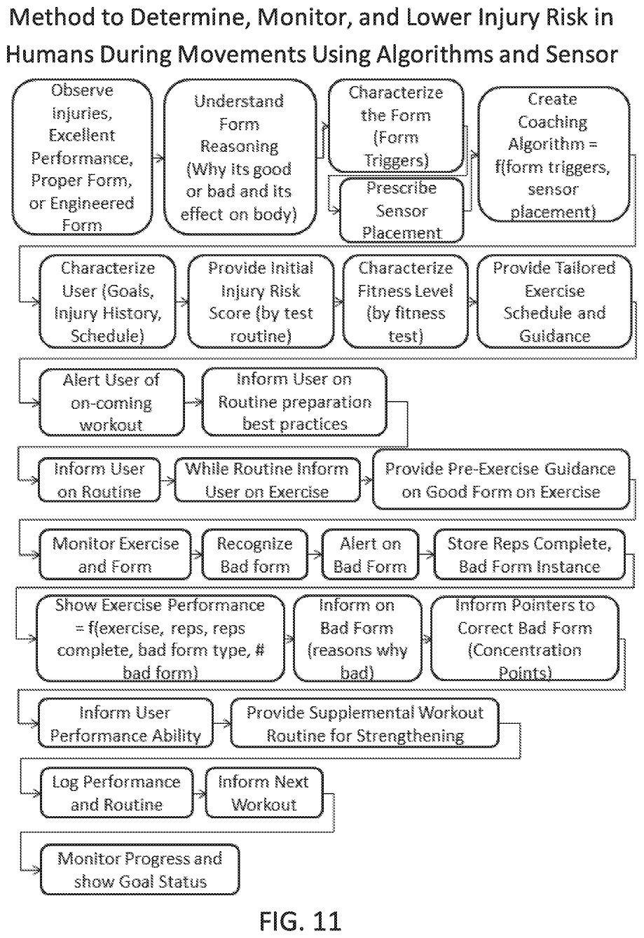

FIG. 11 illustrates a block diagram of a disclosed method of determining and monitoring lower injury risk in humans using algorithms and a sensor configuration, in accordance to an embodiment of the invention.

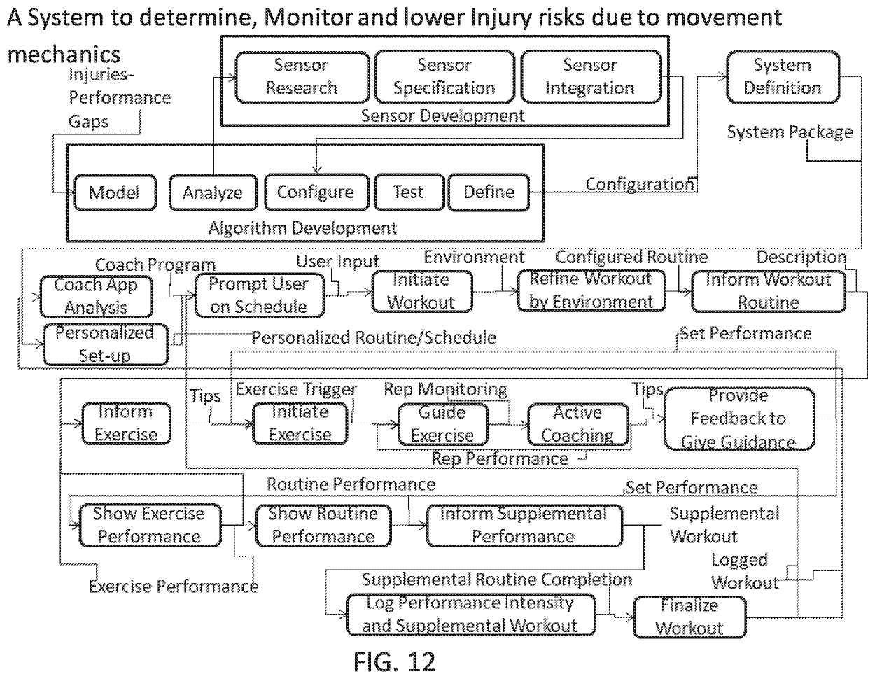

FIG. 12 is a block diagram illustrating a system for determining and monitoring lower injury risks due to movement mechanics, in accordance to an embodiment of the invention. In one disclosed embodiment, the aforementioned system may be implemented, for example, in a fitness application including sensor integration in an electronic device.

The system, as described in the present technique or any of its components, may be embodied in the form of a an electric device such as a computer system. Typical examples of a computer system includes a general-purpose computer, a programmed micro-processor, a micro-controller, a peripheral integrated circuit element, and other devices or arrangements of devices that are capable of implementing the steps that constitute the method of the present technique.

The computer system comprises a computer, an input device, a display unit and/or the Internet. The computer further comprises a microprocessor. The microprocessor is connected to a communication bus. The computer also includes a memory. The memory may include Random Access Memory (RAM) and Read Only Memory (ROM). The computer system further comprises a storage device. The storage device can be a hard disk drive or a removable storage drive such as a floppy disk drive, optical disk drive, etc. The storage device can also be other similar means for loading computer programs or other instructions into the computer system. The computer system also includes a communication unit. The communication unit allows the computer to connect to other databases and the Internet through an I/O interface. The communication unit allows the transfer as well as reception of data from other databases. The communication unit may include a modem, an Ethernet card, or any similar device which enables the computer system to connect to databases and networks such as LAN, MAN, WAN and the Internet. The computer system facilitates inputs from a user through input device, accessible to the system through I/O interface.

The computer system executes a set of instructions that are stored in one or more storage elements, in order to process input data. The storage elements may also hold data or other information as desired. The storage element may be in the form of an information source or a physical memory element present in the processing machine.

The set of instructions may include various commands that instruct the processing machine to perform specific tasks such as the steps that constitute the method of the present technique. The set of instructions may be in the form of a software program. Further, the software may be in the form of a collection of separate programs, a program module with a larger program or a portion of a program module, as in the present technique. The software may also include modular programming in the form of object-oriented programming. The processing of input data by the processing machine may be in response to user commands, results of previous processing or a request made by another processing machine.

All documents, patents, journal articles and other materials cited in the present application are incorporated herein by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus comprising:
a first sensing unit having an output for reading an impact force transmitted to an anatomical joint into a processing unit;
a second sensing unit having an output for reading a flexion angle of the anatomical joint into the processing unit; and
one or more user alert units coupled to the processing unit;
wherein the processing unit computes one or more strain values experienced by the anatomical joint and activates the one or more user alert units when a detrimental strain threshold level is reached.

2. The apparatus of claim 1, wherein the second sensing unit is a variable resistor.

3. The apparatus of claim 1, wherein the first sensing unit is disposed on an anatomical limb connected to the joint.

4. The apparatus of claim 3, wherein the first sensing unit measures a ground reaction force at the anatomical joint upon ground impact.

5. The apparatus of claim 1, wherein the first sensing unit and the second sensing unit are disposed on a sleeve that fits around the joint as a wearable joint injury prevention system.

6. The apparatus of claim 1, wherein an output of the one or more user alert units comprise one or more of visual, auditory, tactile or textual feedback to the user.

7. The apparatus of claim 1, wherein the output of the first sensing unit and the output of the second sensing units are configured to a portable computing unit.

8. The apparatus of claim 7, wherein the portable computing unit is one of a smart phone, smart watch or an electronic tablet.

9. The apparatus of claim 1, wherein the anatomical joint is a knee joint.

10. The apparatus of claim 9, wherein the first sensing unit, the second sensing unit, the processing unit and the one or more user alert units are attached to a knee sleeve which fits around a knee joint as a wearable knee joint injury prevention system.

11. The apparatus of claim 9, wherein the shear strain is the proximal tibia anterior shear force.

12. The apparatus of claim 11, wherein the proximal tibia anterior shear force is experienced during a vertical stop-jump.

13. A method comprising:
measuring an instantaneous momentum of an anatomical limb at an instance of ground impact;
measuring a flexion angle of an anatomical joint coupled to the anatomical limb at the instance of ground impact;
calculating one or more strain values experienced by the anatomical joint using as parameters the speed of the anatomical limb and the flexion angle of the anatomical joint at the instance of ground impact;
alerting a user when the experienced shear strain equals or exceeds a detrimental threshold level.

14. The method of claim 13, further comprising attaching one or more sensors to a sleeve that fits around the joint and using the one or more sensors for measuring the speed of the anatomical limb and the flexion angle of the joint at the instance of ground impact.

15. The method of claim 14, further comprising attaching a processing unit to the sleeve for calculating a shear strain experienced by the joint using one or more outputs of the one or more sensors.

16. The method of claim 13, further comprising transmitting one or more outputs of the one or more sensors to a portable computing device and using an application on the portable computing device for calculating a shear strain experienced by the joint and alerting the user when the experienced shear strain equals or exceeds a detrimental threshold level.

17. The method of claim 16, wherein alerting the user comprises generating one or more of visual, auditory, tactile or textual alerts.

18. The method of claim 16, wherein the portable computing device is one of a smart phone, smart watch or an electronic tablet.

19. The method of claim 18, further comprising providing a user interface for configuring and customizing a functionality of the application on the portable computing device.

20. The method of claim 18, further comprising generating a user evaluation report based on one or more calculations of the shear strain value by the application.
